(19) United States
(12) Patent Application Publication
(10) Pub. No.: US 2005/0070937 A1
(43) Pub. Date: Mar. 31, 2005

(54) Segmented gastric band

(76) Inventors: Kristin L. Jambor, Cincinnati, OH (US); Randal T. Byrum, Milford, OH (US)

Correspondence Address: FROST BROWN TODD, LLC, 2200 PNC CENTER, 201 E. FIFTH STREET, CINCINNATI, OH, 45202, US

(21) Appl. No.: 10/676,946

(22) Filed: Sep. 30, 2003

(51) Int. Cl. A61B 17/08

(52) U.S. Cl. 606/153

(57) ABSTRACT

A stoma-adjustable gastric band having a plurality of chambers within a balloon portion is described. Each chamber is supplied fluid via a corresponding inlet in a fluid supply tube. The balloon and fluid supply tube are each attached to a tension carrying belt. Alternatively, the gastric band may have a plurality of balloons attached to the tension carrying belt where each balloon is supplied fluid via a corresponding inlet in a fluid supply tube.

### BACKGROUND OF THE INVENTION

### FIELD OF THE INVENTION

The present invention generally relates to a device for the treatment of morbid obesity. More particularly, the present invention relates to an easily insertable and removable gastric banding device that encircles and compresses a portion of the stomach thereby forming a stoma opening having a reduced diameter.

Over the years many methods of treating morbid obesity have been undertaken. One of the more promising methods employs the placement of a circumscribing band around a portion of the stomach whereby the stomach may be compressed thereby creating a stoma opening that is smaller than the normal interior diameter of the stomach thereby restricting food intake into the lower digestive portion of the stomach.

Such a band has been described by Kuzmak et al. in U.S. Pat. No. 4,592,339. Kuzmak teaches a stoma-adjustable gastric band that includes a balloon section that is expandable and deflatable through a remote injection site. The balloon expandable section adjusts the size of the stoma opening both intraoperatively and post-operatively.

During the last several years, manufacturers of prior art bands have improved the designs of the balloons of these bands. One significant area of further improvement, however, is the development of a stoma-adjustable gastric band that includes a balloon section that conforms to the patient's anatomy even better than prior art bands. Such an improvement will further assure that the balloon fills uniformly, and that no portion of the balloon wall is highly stressed.”

### SUMMARY OF THE INVENTION

The present invention overcomes the above noted and other deficiencies in the prior art by providing a stoma-adjustable gastric band which is configured such that it will not fold or crease and, as a result, that will reliably and substantially fill with a filling solution.

In one embodiment of the invention, the gastric band generally comprises a tension carrying belt having a fluid supply tube and a balloon disposed thereon. The balloon has a plurality of inner chambers. The fluid supply tube is in fluid communication with the inner chambers of the balloon.

In another embodiment of the invention, the gastric band comprises a tension carrying belt having a fluid supply tube and a plurality of balloons disposed thereon. The fluid supply tube has a plurality of inlets. The fluid supply tube is in fluid communication with each of the balloons by way of the plurality of corresponding inlets.

In yet another embodiment of the invention, the gastric band similarly comprises a tension carrying belt having a fluid supply tube and a balloon disposed thereon. The balloon of this embodiment has a reinforced section located atop the interior wall of the balloon. The balloon is thus adapted to bend between the reinforced sections when the band is placed around the stomach.

The above summary of the present invention is not intended to describe each embodiment or every implementation of the present invention. Advantages and attainments, together with a more complete understanding the invention, will become apparent and appreciated by referring to the following detailed description and claims taken in conjunction with the accompanying drawings.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 presents a pictorial view of a prior art gastric band and associated injection port;

FIG. 2 is a cross-sectional view taken along line 2-2 in FIG. 1;

FIG. 3 is an isometric view of a removable gastric band embodying the present invention;

FIG. 4 is a cross-sectional view taken along line 4-4 of FIG. 3;

FIG. 5 is a cross-sectional view taken along line 6-6 of FIG. 4;

FIG. 6 is a partial cross-sectional view similar to the cross-sectional view of FIG. 5 showing an alternate location for the fluid supply tube;

FIG. 7 is a cross-sectional view of an alternate embodiment of the fluid supply tube having a one-way check valve in the inlet of the fluid supply tube; and

FIG. 8 is a side view of an alternate embodiment of a gastric band including a partial cut away of the balloon portion showing a cross-sectional view of a reinforced section thereof.

### DETAILED DESCRIPTION OF THE INVENTION

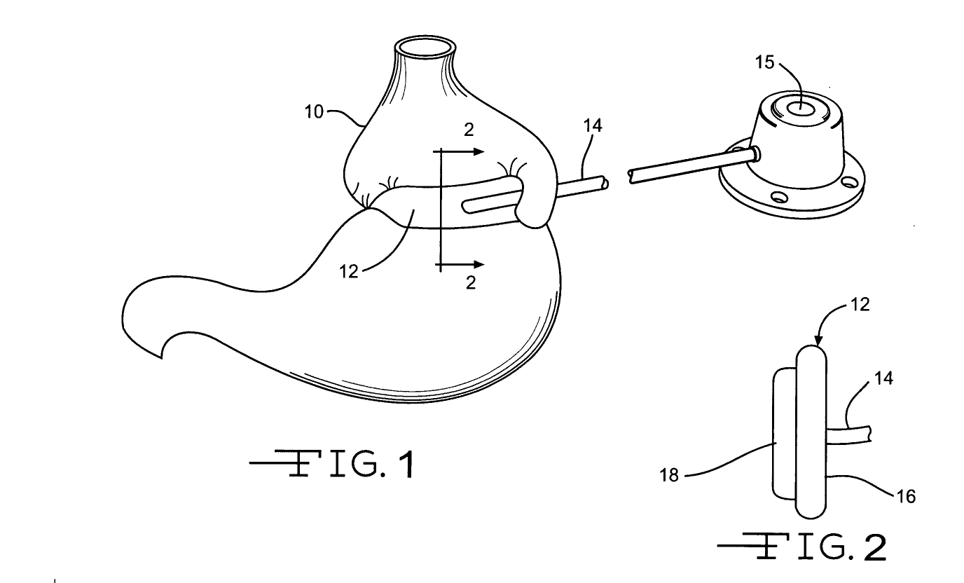

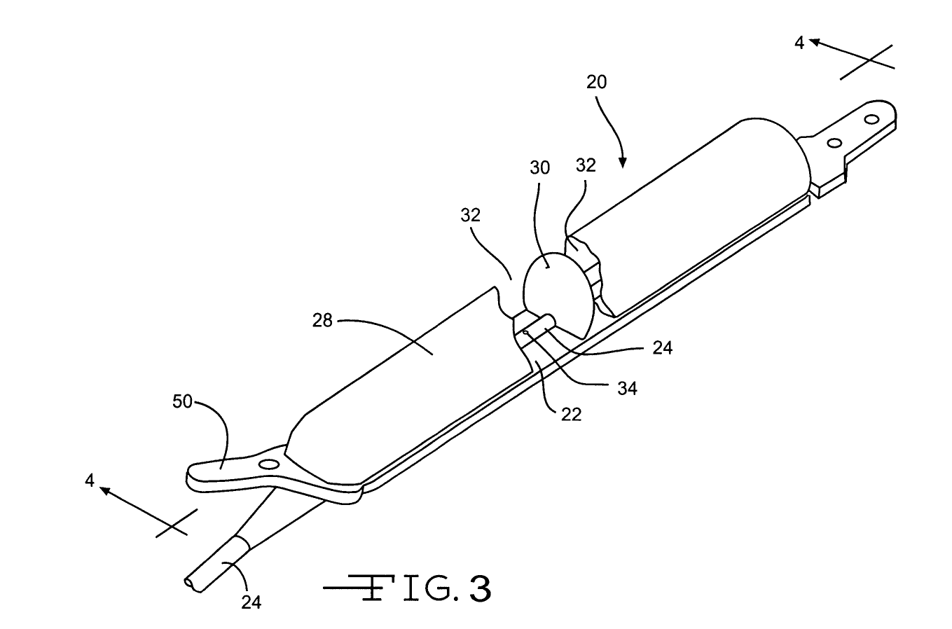

Referring now to the Figures wherein like numerals indicate like elements throughout, FIGS. 1 and 2 depict a stomach 10 having a fluidly inflatable, circumscribing band 12 as is known in the art. A fluid supply tube 14, fluidly communicating with band 12 is in fluid communication with a remotely located fluid injection port 15. Band 12 generally comprises an outer tension carrying belt having an inflatable balloon 18 affixed to the inside thereof. Balloon 18, when inflated, restricts the volume of stomach 10. To inflate balloon 18, a filling solution is injected into injection port 15 and the filling solution is conveyed to balloon 18 by way of supply tube 14.

The balloon 18 of the prior art band 12 has a tendency to fold and create creases when the band 12 is placed around the stomach 10. These creases may, in some instances, restrict flow of the filling solution to all areas of the balloon 18. This will affect the geometry of the balloon 18 and may also damage the balloon 18.

Referring now to FIGS. 3 through 5, the segmented gastric band 20 of the present invention is shown. Similar to prior art gastric bands, the segmented gastric band 20 has a tension carrying belt 22, a fluid supply tube 24 in fluid communication with a balloon 28 and a remotely located fluid injection port (not shown). The balloon 28 is preferably of a length of about 8 cm to about 15 cm and more preferably about 11 cm; however, it should be appreciated that the balloon 28 may be of any length which would provide sufficient compression of the stomach. The balloon 28 is preferably comprised of material with a thickness between about 0.3 cm and 0.7 mm and more preferably about 0.5 mm. Obviously, the thickness of the material is dependent on the balloon material and it should be appreciated that the thickness of the balloon may vary depending on the balloon composition. The tension carrying belt 22 is preferably longer than the balloon 28 and may be of any suitable length sufficient to accommodate the type of latching mechanism 50 employed. All components of segmented gastric band 20 are preferably comprised of medical grade silicone polymer but may be comprised of any flexible biocompatible material including implantable polyurethane.

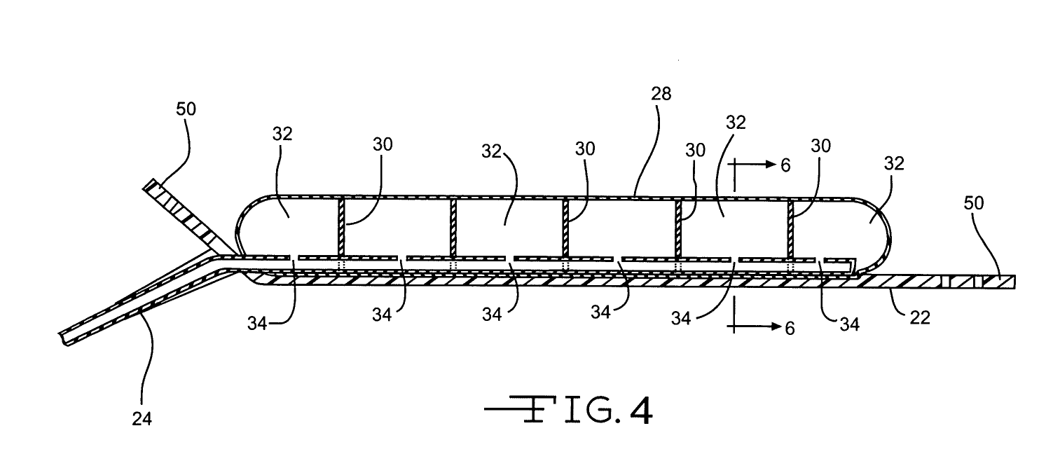

As best shown in FIG. 4, the segmented gastric band 20 also has one or more partitions 30 located inside balloon 28, which separate the interior volume of balloon 28 into one or more chambers 32. The partitions may be evenly spaced within the balloon (as shown), unevenly spaced within the balloon, perpendicular to the longest edge of the tension carrying belt 22 or at an angle relative to the longest edge of the tension carrying belt 22. Also, partitions 30 may be reinforced with alternate material configurations or features such as multiple layers including one or more materials, thicker material, textured material, or a more dense material so as to help the balloon 28 bend at these locations.

Fluid supply tube 24 has inlets 34 each of which correspond with and distribute the filling solution to each chamber 32. Inlets 34 may be of substantially identical diameters. Alternatively, inlets 34 may be ordered along the tube 24 from smallest diameter to largest diameter from end closest to fluid injection port such that the chamber closest to injection port fills with the filling solution at substantially the same rate as chambers further from the injection port. Also, in some applications, and as shown in FIG. 7, it may be beneficial to place a one way check valve in inlet 34 such that the filling solution is allowed to enter the chamber but is not allowed to leave chamber via inlet 34. For example, FIG. 7 shows fluid supply tube with inlet 34 including a duck bill type check valve 36 whereby the filling solution is allowed to enter chamber but is not allowed to escape via inlet 34. While a duck bill type check valve is shown in the illustrative embodiment, any type of one way check valve which would allow the filling solution to enter chamber and prevent the filling solution from escaping chamber via inlet would suffice. Alternatively, since, on occasion, the filling solution may need to be removed from the balloon, it may be advantageous to use a two-way check valve where the two-way check valve only allows the filling solution to leave the chambers when the pressure in the fluid supply tube becomes less than the pressure of the filling solution in the chambers. Preferably, this pressure differential would be created by using a syringe to withdraw fluid through the injection port.

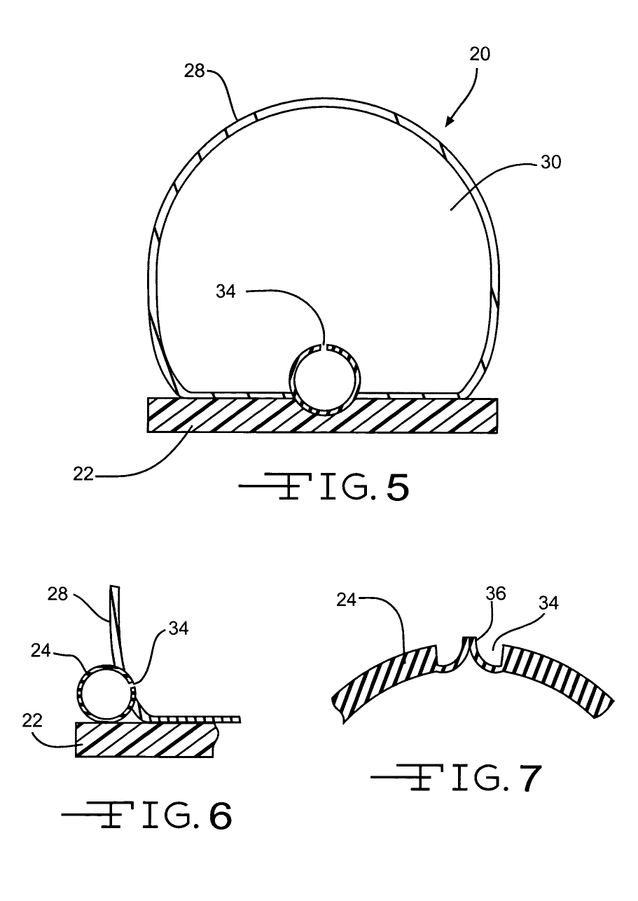

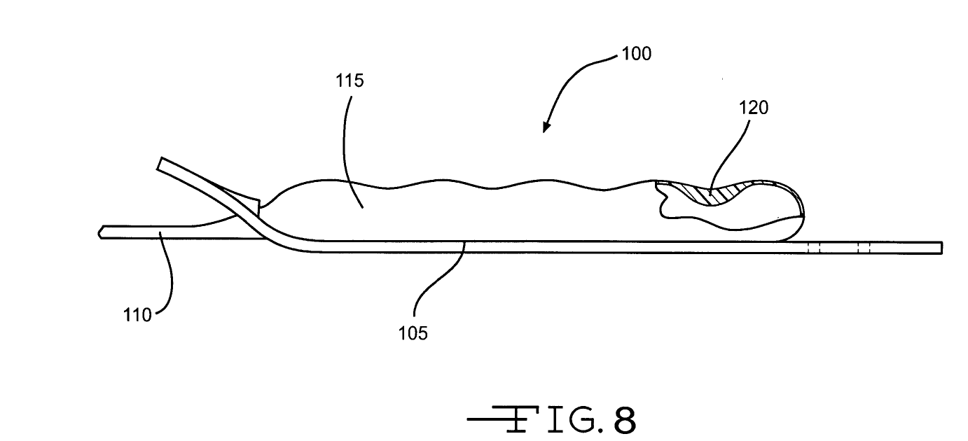

The fluid supply tube 24 may be attached atop the surface of belt portion 22 inside balloon 28, or fluid supply tube 24 may be imbedded in belt portion 22 inside balloon 28, as shown in FIG. 5. Fluid supply tube 24 may also be attached atop the surface of belt portion 22 outside of balloon 28, as shown in FIG. 6, or fluid supply tube 24 may be imbedded in belt portion 22 outside of balloon 28.

Alternatively, each of the partitions may have a small opening which would allow the filling solution to flow from one chamber to the next via the opening. In this case, the fluid supply tube would only need an inlet to one chamber, preferably one of the chambers at either end of the balloon; although, multiple inlets could still be used.

The segmented gastric band 20 also includes a latching mechanism 50 so that the segmented gastric band may be releasably secured in an encircled position around a portion of the stomach. Latching mechanism 50 may be of any suitable configuration to hold segmented gastric band 50 in an encircled position such as, but not limited to, a guide tab and buckle configuration, a slide and channel configuration, a hook and eye configuration or, as shown in FIGS. 3, 4 and 8, tabs which are sutured together.

Installation of the segmented gastric band 20 is accomplished by first inserting the band into the patient's abdomen through a trocar. Next, a tunnel is created behind the stomach near the esophagogastric junction using a blunt dissection device. The segmented gastric band 20 is then grasped by an instrument, such as a grasper or blunt dissection device, and wrapped around the patient's stomach through the created tunnel. The latching mechanism 50 is then engaged. The injection port is then attached to the gastric band and the injection port is secured subcutaneously in the abdomen or other suitable location. A suitable filling solution, such as saline, is then injected into injection port whereby the solution is conveyed to chambers 32 of balloon 28 by way of inlets 34 in fluid supply tube 24. If necessary either at the time the gastric band is installed or at some time in the future, a predetermined quantity of the filling solution may be withdrawn for the balloon 28 by inserting a syringe into the injection port and withdrawing the solution.

An alternative embodiment of the gastric band of the present invention is shown in FIG. 8. In this embodiment the gastric band 100 similarly includes a tension carrying belt 105, a fluid supply tube 110 in fluid communication with a balloon 115 and a remotely located fluid injection port (not shown). In this embodiment, the balloon 115 has one or more reinforced sections 120. Preferably, the reinforced sections 120 are spaced evenly along the interior length of the balloon 115. Reinforced sections 120 may be comprised of a thicker material, textured material, a harder or softer material, or other similar configurations that would help balloon 115 bend between adjacent reinforced sections 120 when placed in an encircling position around stomach. In the preferred embodiment, shown in FIG. 8, each end of each reinforced section 120 is substantially the same thickness as the balloon 115; however, each reinforced section is thickest, and thicker than the balloon, at its midpoint. At the midpoint, each reinforced section 120 is preferably between about two and eight times the thickness of the balloon 115, and more preferably about four times the thickness of the balloon 115. Reinforced sections 120 may or may not be used to cause balloon 115 to segment into chambers when encircled around stomach.

While the present invention has been illustrated by the description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, instead of using partitions in balloon to create chambers in balloon, a segmented gastric band may be comprised of a plurality of balloons spaced along and attached to tension carrying belt.

Further, it will become readily apparent to those skilled in the art that the above invention has equally applicability to other types of implantable bands. For example, bands are used for the treatment of fecal incontinence. One such band is described in U.S. Pat. No. 6,461,292 which is hereby incorporated herein by reference. Bands can also be used to treat urinary incontinence. One such band is described in U.S. patent application 2003/0105385 which is hereby incorporated herein by reference. Bands can also be used to treat heartburn and/or acid reflux. One such band is described in U.S. Pat. No. 6,470,892 which is hereby incorporated herein by reference. Bands can also be used to treat impotence. One such band is described in U.S. patent application 2003/0114729 which is hereby incorporated herein by reference.

1. A gastric band comprising:
a tension carrying belt;
a balloon attached to said tension carrying belt, said balloon comprising a plurality of partitions defining a plurality of inner chambers; and
a fluid supply tube in fluid communication with said balloon and attached to said tension carrying belt.

2. The gastric band of claim 1, wherein said fluid supply tube further comprises a plurality of inlets wherein each of said plurality of inlets is in fluid communication with a corresponding one of said plurality of inner chambers of said balloon and said fluid supply tube.

3. The gastric band of claim 2, wherein said fluid supply tube further comprises a plurality of check valves each of which is located within one of said plurality of inlets.

4. The gastric band of claim 3, wherein each of said plurality of check valves comprises a one-way check valve.

5. The gastric band of claim 3 wherein each of said plurality of check valves comprises a two-way check valve.

6. The gastric band of claim 2, wherein two or more of said plurality of inlets of said fluid supply tube are of different sizes.

7. The gastric band of claim 6, wherein said plurality of inlets are ordered from smallest to largest along said fluid supply tube such that said plurality of chambers of said balloon fill at relatively substantially similar rates.

8. The gastric band of claim 1, wherein said balloon has a length between about 8 cm and about 15 cm.

9. The gastric band of claim 1, wherein said tension carrying belt further comprises a latching mechanism.

10. The gastric band of claim 9, wherein said latching mechanism comprises one or more sutures.

11. A gastric band comprising:
a tension carrying belt;
a plurality of balloons wherein said plurality of balloons is attached to said tension carrying belt; and
a fluid supply tube comprising a plurality of inlets wherein said fluid supply tube is attached to said tension carrying belt such that said plurality of inlets of said fluid supply tube provide fluid communication between each of said plurality of balloons and said fluid supply tube.

12. The gastric band of claim 11, wherein said fluid supply tube further comprises a plurality of check valves each of which is located within one of said plurality of inlets of said fluid supply tube.

13. A gastric band comprising:
a tension carrying belt having a top portion;
a balloon having an interior surface, said balloon further comprising one or more reinforced sections disposed along the length of said interior surface of said balloon said balloon being attached to said tension carrying belt; and
a fluid supply tube wherein said fluid supply tube provides fluid communication between said balloon and said fluid supply tube.

14. The gastric band of claim 13, wherein upon being placed in an encircling position around a stomach, each of said reinforced sections contacts said top portion of said tension carrying belt dividing said balloon into at least two chambers.

15. The gastric band of claim 14, wherein said fluid supply tube further comprises a plurality of inlets wherein each of said plurality inlets of said fluid supply tube is in fluid communication with at least one of said at least two chambers of said balloon and with said fluid supply tube.

16. The gastric band of claim 15, wherein said fluid supply tube further comprises a plurality of check valves each of which is located within one of said plurality of inlets of said fluid supply tube.

17. The gastric band of claim 16, wherein said balloon has a length between about 8 cm and about 15 cm.

18. The gastric band of claim 17, wherein said one or more reinforced sections comprise a material thicker than balloon.

19. The gastric band of claim 18, wherein each of said one or more reinforced sections has a first end, a second end and a midpoint wherein the thickness of each of said one or more reinforced sections at said first end and said second end is substantially the same thickness as said balloon and wherein said thickness of each of said one or more reinforced sections at said midpoint is greater than the thickness of said balloon.

20. The gastric band of claim 19, wherein the thickness of each of said one or more reinforced sections at said midpoint is between about two and about eight times the thickness of said balloon.
